(19) United States
(12) Patent Application Publication
(10) Pub. No.: US 2011/0162113 A1
(43) Pub. Date: Jun. 30, 2011

(54) ANTIBODIES

(75) Inventors: Lorenzo FRIGERIO, Coventry (GB); Jane Hadlington, Coventry (GB)

(73) Assignee: University of Warwick, Coventry (GB)

(21) Appl. No.: 12/830,214

(22) Filed: Jul. 2, 2010

Related U.S. Application Data
(63) Continuation of application No. 10/535,433, filed on Feb. 2, 2006, now abandoned.

(30) Foreign Application Priority Data
Nov. 18, 2002 (GB) 0226878.7

(51) Int. Cl. C12P 21/00; C12P 21/02; C12P 21/06; C07K 16/00; A61K 39/395; C12N 15/63; A01H 5/00; G01N 33/566; C12N 5/10

(52) U.S. Cl. 800/295; 435/69.6; 435/68.1; 530/387.3; 424/133.1; 435/320.1; 436/501; 435/419; 435/328

(57) ABSTRACT

The application discloses a method of making an antibody molecule, the antibody containing an immunoglobulin heavy chain comprising a α3 domain or a mu domain, the method comprising: (a) Providing a nucleotide sequence encoding the immunoglobulin heavy chain; (b) Modifying the nucleotide sequence in the region of the nucleotide sequence encoding the C-terminus 18 amino acids of the completed heavy chain to remove, or reduce the effectiveness of, one or more vacuolar targeting signal sequences to form a modified nucleotide sequence; (c) Inserting the modified nucleotide sequence into a host cell; and (d) Causing the host cell to express the modified nucleotide sequence to form the modified antibody heavy chain and secrete the modified antibody heavy chain from the host cell. This improves the secretion of the antibody from, for example, plant cells. Methods of adding J-chain binding activity to antibodies are also provided. The modified antibodies and their use is also disclosed.

The invention relates to methods of improving the secretion of antibodies from the cells of organisms, such as plants and antibodies having heavy chains which are modified at the C-terminus ends in order to improve their secretion from a cell expressing the antibody. Modifications to the C-terminus ends of heavy chains of antibodies to allow binding of the heavy chain to J-chains are also included in the invention.

Plants are an efficient and versatile system to express foreign proteins and can even produce complex, multimeric proteins in large amounts. One such example is the expression of a decameric, secretory immunoglobulin known as IgA, a molecule composed of two IgA units (containing 2 heavy and 2 light chains), a joining J chain and a secretory component. It has been shown that transgenic tobacco cells are able to translocate all the IgA subunits in the endoplasmic reticulum (ER), where complex assembly occurs with high efficiency. The overall production of IgA is very high, making plants the system of choice for the expression of these molecules.

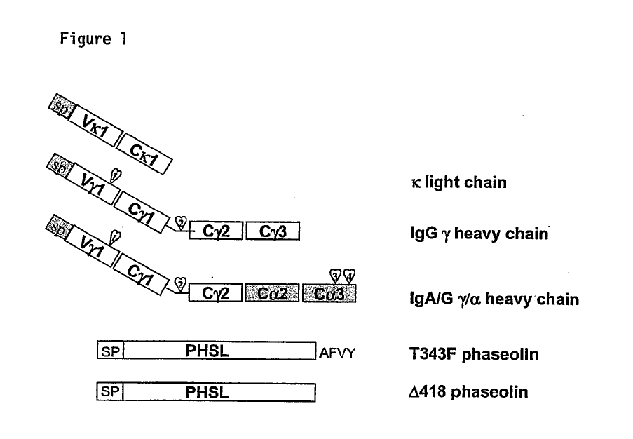

U.S. Pat. No. 6,417,429 and related International Patent Application No. WO 97/42313 (Scripps) disclose the expression of multimeric proteins, such as antibodies in plants. IgA is a secretory type of antibody. It contains a domain at the C-terminal end of each of its heavy chains (the Cα3 domain, see for example FIG. 1 of the current application) which contains a cysteine residue which binds the peptide, known as the J chain. Naturally occurring IgA exists as two monomeric units (a unit is defined as a tetramer composed of two heavy chains and two light chains) of IgA antibodies joined by the J chain, in combination with a fourth component, a polypeptide known as the secretory component. This produces a complex which is resistant to degradation caused by proteases present in mucosal environments such as the oral cavity.

The Cα3 domain has been incorporated into, for example, the heavy chains of IgG antibody heavy chains (which cannot naturally bind J chains) to form IgA/G hybrids capable of binding J chains. This improves the stability of the antibody without altering antigen binding or specificity.

Methods of expressing antibodies are discussed in detail in U.S. Pat. No. 6,417,429 and WO 97/42313. The United States patent discloses the prediction of plant cells containing nucleotide sequences encoding immunoglobulin heavy- and light-chain polypeptides, each polypeptide containing a leader sequence forming a secretion signal and immunoglobulin molecules encoded by the nucleotide sequences where the leader sequences cleaved from the immunoglobulin molecules following proteolitic processing in the plants. The method for producing such transgenic plants is achieved, for example, by introducing into the genome of a first member of the plant species a first mammalian nucleotide sequence encoding an immunoglobulin heavy chain containing polypeptide including a leader sequence forming a secretion signal to produce a first transformed cell. A second mammalian nucleotide sequence encoding an immunoglobulin light chain containing polypeptide including a leader sequence forming a secretion signal is then inserted into the genome of a second plant species to produce a second transformant. The two transformed plants are then cross-pollinated to create progeny. Transgenic plant species are isolated from the progeny which are capable of producing an immunoglobulin molecule, where the leader sequence is cleaved from the immunoglobulin molecules by proteolytic processing to produce the completed antibody. The completed antibody typically comprises four polypeptide chains, two identical light chains (L) and two identical heavy chains (H). The four chains are held together by a combination of non-covalent interactions and covalent bonds. The molecule is typically composed of two identical halves in which both L and H chains contribute almost equally to the two identical antigen binding sites.

IgM is another major class of antibody. This is secreted into the blood in the early stages of a primary antibody response. In the secreted form IgM is a pentamer comprised of five 4-chain units and thus has a total of ten antigen-binding sites. Such pentamers contain a copy of a J chain polypeptide joining the C-terminus end of the heavy chains of two adjoining 4-chain units.

The inventors have recently discovered that not all the assembled antibody, whose fate is secretion in mammals, is secreted by plant cells. Rather, a high proportion of the molecules are retained intracellularly and eventually delivered to vacuoles, where they are ultimately degraded. This transport and subsequent degradation in the vacuolar compartment can be inhibited by treatment with the fungal metabolite Brefeldin A and all IgA assembly intermediates—from the decamer to the simplest, heterotetrameric unit—share the same intracellular fate. In contrast, when the parent IgG molecule was expressed, it was efficiently secreted. As the kappa light chain is the same for both IgG and IgA/G molecules, this led the inventors to speculate that features of the IgA/G heavy chain might be responsible for this intracellular diversion. Alternatively, the nature of the heavy chain could impose stress on the endoplasmic reticulum, causing the subsequent missorting to the vacuole.

The Frigerio paper does suggest that the α-domains present in the hybrid IgA/G heavy chains might be involved in some way in intracellular retention and vacuolar delivery. However, the paper highlighted a wide variety of potential areas which looked as though they could be involved in intracellular retention and vacuolar delivery.

Firstly, there was the possibility that the endoplasmic reticulum was stressed by the presence of the recombinant protein. There was the potential that this stress caused the endoplasmic reticulum to reject the protein in some way. The inventors have now proved that this is not the case, as the endoplasmic reticulum of these plants can cope with the co-expression of a further foreign protein without disposing of it.

There was the further possibility that α-domains cause structural defects which lead to endoplasmic reticulum retention and eventual disposal in vacuole. The inventors have now demonstrated that this is not the case as the vacuolar fragments derived from assembled sub-units, and the immunoglobulin would not assemble if it had structural defects.

The paper also suggested that cysteine residues were in some way involved. In mammalian cells, exposed cysteine residues in the C-terminal region of the heavy chains of IgA and secretory IgM, are recognised by endoplasmic reticulum quality control. The observations in the paper, that the parent IgG tetramers, which do not have free cysteines, are secreted with high efficiency, was thought to be consistent with the possibility that thiol-mediated retention also occurs in plant ER. This strongly suggested that cysteine residues on the IgA/G units were involved in the retention. This has now been proved by the inventors not to be the case as treatment with the reducing agents does not cause more secretion. The treatment of reducing agents in mammals has been previously observed to increase secretion of proteins.

The paper also suggested that the Cα2 domain was involved. The hybrid immunoglobulins contain extra Cα2 domains. The paper suggests that extra Cα2 domains in the heavy chain might affect interactions with chaperones or recognition by quality control mechanisms in the endoplasmic reticulum. Again, the inventors have now identified that the Cα2 domain is not involved in intracellular retention and vacuolar delivery.

The inventors therefore looked at the Cα3 domain. This is a domain of approximately 120 amino acid residues. The inventors have now unexpectedly found that the last 18 amino acid residues at the C-terminus end are involved in intracellular retention and vacuolar delivery. Furthermore, they have identified that this particular region also has high levels of homology with IgM and they are therefore likely to be involved in the retention of recombinant antibodies containing IgM-derived heavy chains.

Furthermore, the inventors have realised that simply deleting the last 18 amino acids from the C-terminal end of such antibodies improves secretion, it also results in the removal of the residue required for binding of the antibody to the J-chain polypeptide. This removed the ability to generate multimeric antibody molecules. The inventors have therefore identified a method of reinstating the ability of the recombinant antibody to bind J-chain polypeptides. The latter method may also be used to introduce J-chain binding capability to non-IgA or IgM derived antibodies.

Accordingly, the first aspect of the invention provides a method of making an antibody molecule, the antibody containing a heavy chain comprising an α3 domain or a mu domain, the method comprising:

(a) providing a nucleotide sequence encoding the immunoglobulin heavy chain;

(b) modifying the nucleotide sequence in the region of the nucleotide sequence encoding the last 18 amino acids of the completed heavy chain to remove, or reduce the effectiveness of, one or more targeting signal sequences to form a modified nucleotide sequence;

(c) inserting the modified nucleotide sequence into a host cell; and

(d) causing the host cell to express the modified nucleotide sequence to form the modified heavy chain and secrete the modified antibody heavy chain from the cell.

The α3 domains are derived from immunoglobulins classed as immuno globulin A (IgA chains). Mu domains are derived from immunoglobulin M (IgM heavy chains). The heavy chains may be from IgA or IgM heavy chains or may be hybrids of other classes of immuno globulins which have had an α3 domain or a mu domain added to their heavy chain. Such hybrids include the known IgA/G hybrids.

Typically, the heavy chain is co-expressed with a light chain within the cell and is secreted as a tetramer containing two heavy chains and two light chains.

The modified antibody molecule has improved secretion compared with non-modified antibody.

The nucleotide sequence may be, for example, a DNA or RNA molecule encoding the immunoglobulin heavy chain.

The nucleotide sequence modified comprises one or more of the nucleotides which encode the last 18 amino acids of the completed heavy chain. That is, from the C-terminal end of the heavy chain once the secreted antibody has been exported from the host cell and optionally further processed to form the completed antibody molecule. The completed antibody molecule may be in the form of separate heavy chains. More preferably, the completed antibody molecule consists of four polypeptide chains, two light chains and two heavy chains, the heavy chains of which comprise an α3 domain or a mu domain which has been modified. Two or more of the four polypeptide units may be in turn joined together, for example to form a dimeric IgA molecule containing a J-chain or, for example, a pentameric IgM molecule which also comprises a J-chain.

The modification of the nucleotide sequence may be via one or more mutations to alter the amino acid sequence for which the nucleotide sequence encodes. This may be via one or more point mutations, deletions, additions or replacement of one or more of the nucleotides with alternative nucleotides. Preferably nucleotides encoding 1, 2, 3, 4, 5, 6, 7, 8, 9, 10, 15, 16, 18 or more of the C-terminal amino acids are deleted as a result of modifying the nucleotide sequence encoding them. The alteration causes the amino acid sequence of targeting signal to be changed so that it is no longer recognised by the host cell, for example, as a vacuolar sorting signal.

The review article by Matsuoka K. and Neuhaus J. M., (J. Exp. Bot. Vol. 50 (1999), pages 165-174) suggests that the function of C-terminal vacuolar sorting signals can be decreased, and even abolished, by adding two to four glycine residues downstream of the sorting signal. The inventors of the present application expect that addition of at least two, preferably two to four, glycine residues downstream of a C-terminal sorting signal of an antibody will have a similar affect to deleting or mutating the sorting signal, that is: it will result in the decrease or abolition of vacuolar sorting of the antibody. Alternative ways of ‘masking’ the C-terminal sorting signal are envisaged, e.g. other ‘inocuous’ amino acids, e.g. Asparagine or Alanine may be located downstream of the C-terminal sorting signal. Such C-terminal extensions can arise easily without affecting the proper folding and activity of proteins.

Accordingly, preferably the region encoding the C-terminus is modified by the addition of nucleotides encoding 1 or more, typically 2 to 8, especially 2 to 4 amino acid residues, such as glycine, asparagine or alanine. This modification may be downstream of the C-terminal sorting signal.

Amino acids are encoded by codons of three nucleotides. The codons encoding each amino acid are conserved between organisms. Changing one or more nucleotides in a codon for a different nucleotide can change the amino acid encoded by the codon.

The targeting signal preferably encodes one or more amino acids which inhibit the secretion of the heavy chain polypeptide from the cell. This may be a signal which inhibits secretion from the endoplasmic reticulum and/or from a vacuole within a cell.

It is expected that such sequences are likely to affect secretion from the cells of organisms such as higher eukaryotic animals including mammals, insects and crustacea, in addition to plant cells. Such organisms are thought to have sorting machinery that could potentially misinterpret amino acid sequences present on a foreign protein.

Accordingly, preferably the host cell is a higher eukaryotic animal cell or a plant cell. Most preferably the plant cell is a dicotyledonous plant cell, that is from a flowering plant whose embryos have two seed halves or cotyledons. Alternatively, the plant may be a monocotyledonous plant, that is a flowering plant whose embryos have one cotyledon or seed leaf.

0The plant may also be a lower plant, that is any non-flowering plant including ferns, gymnosperms, conifers, horse tails, club mosses, liverworts, hornworts, mosses, red algae, brown algae, gametophytes, sporophytes of pteridophytes, and green algae.

The modified nucleotide sequence may be inserted into the host cell by means of any techniques known in the art. For example, if the host cell is a plant, Agrobacterium-mediated plant transformation, protoplast transformation, gene transfer into pollen, injection into reproductive organs and injection into immature embryos may be used. Additionally, particle guns may be used to introduce the nucleotide material into plant cells. Examples of such methods are described in detail in, for example, U.S. Pat. No. 6,417,429.

Preferably, the host cell is within a mature organism, such as a mature transgenic plant. Alternatively, the host cells may be grown in tissue culture, for example in the form of a callus or suspension of cells.

In the preferred method of making the cells expressing the antibody molecule, a first nucleotide sequence encoding the modified antibody heavy chain is inserted into the genome of a first plant to produce a first transformant. A second nucleotide sequence including an immunoglobulin light chain portion-containing polypeptide is introduced into a second plant to produce a second transformant. The two transformants are then cross-pollinated to produce a progeny population. The transgenic plants are then isolated from the progeny population, the transgenic plants producing an antibody molecule containing a heavy chain with the modified C-terminus.

As already indicated, such methods of producing plants are discussed in, for example, U.S. Pat. No. 6,417,429.

Preferably, the nucleotide sequence encoding the immunoglobulin heavy chain comprises a leader sequence forming a secretion signal. This allows the immunoglobulin heavy chain to be secreted from the cell. The nucleotide sequence encoding the immunoglobulin light chain would also preferably have a secretion signal to allow that light chain to be secreted. Such secretion sequences are normally sheared off after secretion of the proteins from the cell.

The nucleotide sequences also preferably contain one or more control sequences, such as promoters and termination sequences, to allow the production of the heavy chain to be regulated. The promoters may themselves be inducible or constitutive. The promoters may also be temporally regulated, where the promoter is controlled depending on the time during development of the host cell. Additionally, or alternatively, the promoter may be spatially regulated, that is it is regulated depending on where the host cell is within an organism, for example in order to specifically express the heavy chain within the leaves or roots of a plant.

Vectors including the modified nucleotide sequence are also included within the scope of the invention. Vectors are molecules which serve to transfer nucleotides of interest into a cell. Such vectors are well known in the art and will vary depending on the host cell. For example, viral vectors, such as adenoviral or baculoviral vectors may be used with mammalian or insect cells respectively. Alternatively, plasmids based upon the Ti plasmid from Agrobacterium tumefaciens may be used, for example, to introduce material into plant cells.

Host cells and whole transgenic organisms containing the modified nucleotide sequences and/or vectors, are also included within the scope of the invention.

The nucleotide sequences modified to remove the targeting signal, that is, the nucleic acid sequence encoding the targeting signal in the final antibody heavy chain, may be modified by either deletion of that nucleotide sequence or the changing of the nucleic acid sequence so that it no longer encodes for that targeting sequence by the insertion, deletion or changing of one or more nucleic acids within the sequence.

Preferably, the nucleotide sequence modified encodes the amino acid sequence:
X₁ X₂ X₃ V S X₄ where X₁═N, H or L
X₂═V or Y
X₃═S or N
X₄=an aliphatic amino acid, especially V or L.

Preferably, the amino acid sequence is N V S V S V. This has been identified by the inventors as being a targeting signal.

Two other targeting signals have been identified within the C-terminus amino acid sequence of the heavy chain. The inventors have noted that leucine and isoleucine amino acids are present. In other molecules, these two amino acids are typically, sites of glycosylation and are thought to have an effect on the secretion of proteins from cells.

Accordingly, one or more nucleic acids encoding isoleucine or leucine may be modified.

One possible way of modifying the nucleotide sequence is simply deleting nucleotides so that two or more amino acids are deleted from the final modified antibody heavy chain. The inventors have realised that this is potentially disadvantageous. The cysteine residue which is usually found two amino acids from the C-terminus end of the heavy chain, is involved in the binding of J-chain polypeptides. J-chain polypeptides allow the formation of dimeric antibodies or pentemeric antibodies and improve the stability of the secreted antibodies. There is therefore a need to retain or replace the cysteine residue at this position. Accordingly, some of the nucleotides encoding the C-terminus 18 amino acids of the completed heavy chain may be deleted and replaced by a synthetic nucleotide sequence encoding an amino acid sequence and general formula:

(Xaa₁)ₘ C(Xaa₂)ₙ

where: C=a cysteine residue
   Xaa₁=independently any amino acid with the proviso that it is not selected from I, L or forms a consecutive sequence X₁ X₂ X₃ V S X₄
      where: X₁═N, H or L
      X₂═V or Y
      X₃═S or N
      X₄=aliphatic amino acid
   Xaa₂=independently any amino acid
   m=an integer of at least 2
   n=an integer of 0 to 5.

Preferably m=4-20, especially 5 to 10, and n=0 or 1, especially 1. Preferably Xaa₁ is not N V S V S V. Most preferably Xaa₂ is Y or A, especially A.

Such a sequence may also be added to an existing heavy chain sequence to impart J-chain binding capability.

A method of adding J-chain binding ability to the heavy chain of an antibody comprising the steps of:
(a) providing a nucleotide encoding an immunoglobulin heavy chain;
(b) adding to that nucleotide at the position of the nucleotide encoding the C-terminal end of the heavy chain, a nucleotide sequence encoding a synthetic tail with the amino acid sequence:

(Xaa₁)ₘ C(Xaa₂)ₙ

which is defined as above;
(c) expressing the completed nucleotide in a host cell to form immunoglobulin heavy chain capable of binding J-chain.

Preferably, the tail is added at the position of the nucleotide codon encoding the C-terminal end of the final, processed, heavy chain.

The amino acid sequence before the cysteine residue is preferably designed to ensure that the sequence is devoid of any secondary structure so that the cysteine residue is exposed to the medium surrounding the antibody to allow the cysteine residue to efficiently bind to J-chains.

Preferably the modified amino acid is one or both of an isoleucine, 3 amino acids and/or 10 amino acids from the C-terminus end of the completed heavy chain.

Preferably, the nucleotide sequence which is modified is contained within a nucleotide sequence encoding the sequence:

P T X₁ X₂ X₃ V S X₄ X₅ X₆ X₇ X₈ X₉ X₁₀ X₁₁ X₁₂ C Y

where: X₁═N, H or L, preferably N
   X₂═V or Y, preferably V
   X₃═S or N
   X₄=an aliphatic amino acid, preferably V or L
   X₅=an aliphatic amino acid, preferably I, V or L
   X₆=M, V or L, especially M
   X₇═S or A
   X₈=E or D
   X₉=any amino acid, preferably G, V, A or T
   X₁₀=D, E, G or A, preferably D
   X₁₁=G or S, preferably G
   X₁₂═I, T, V, Z or A, preferably I or T.
   X₁₃=may or may not be present and, where present is A or Y, preferably A.

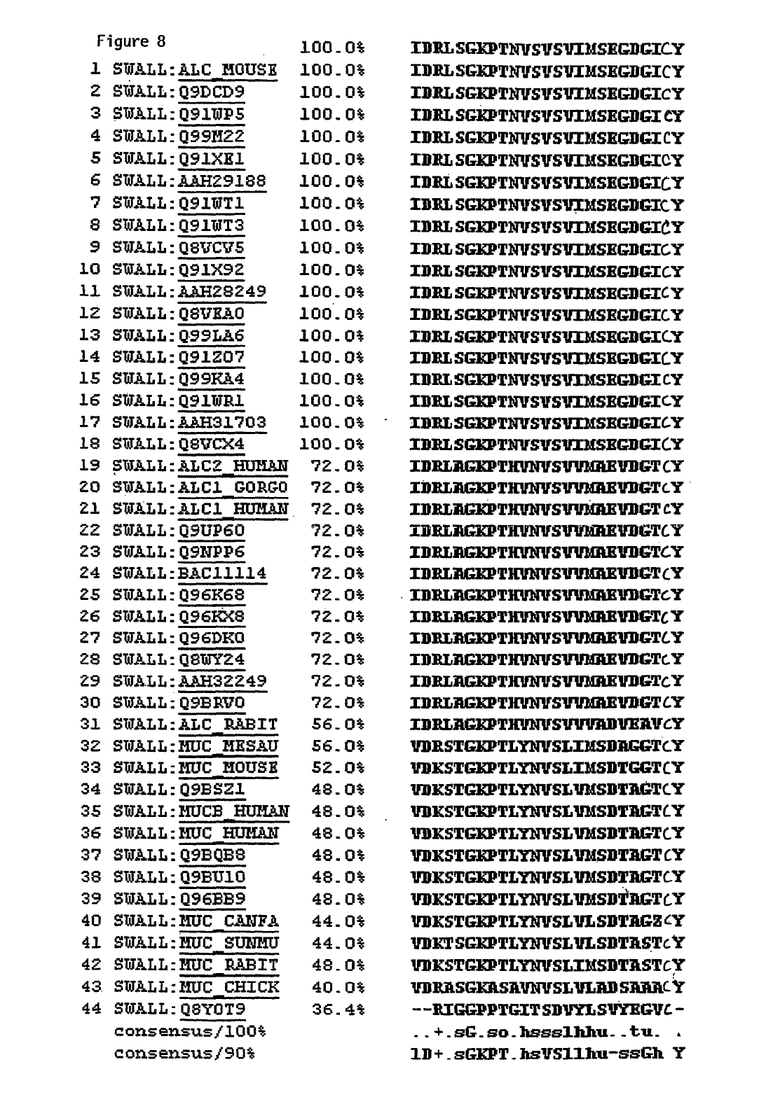

This is based upon a consensus sequence which has been derived from a study of the C-terminus ends of heavy chains from IgA and IgM molecules (see FIG. 8).

The method of the invention additionally comprises the step of isolating and purifying the antibody molecules. The antibody molecules may be harvested and purified by methods known in the art. For example, if the host is a plant cell, then a portion of the transgenic plant may be homogenised to form a plant pulp and the antibody extracted from the pulp.

The extracted antibody may be subjected to a protease digest, for example with papain or pepsin to form Fab or F(ab′)₂ fragments.

Antibodies obtainable by the methods of the invention are also provided.

The method also provides an isolated antibody containing a heavy chain comprising an α3 domain or a mu domain, the α3 domain or mu domain lacking one or more targeting signals towards the C-terminus end. Preferably, the targeting signal is not within the last 18 amino acids of the C-terminus end of the heavy chain.

Preferably, the antibodies of the invention do not contain the targeting signal:
X₁ X₂ X₃ V S X₄
where: X₁═N, H or L
X₂═V or Y
X₃═S or N
X₄=aliphatic amino acid.

Preferably, the targeting signal is N V S V S V.

The antibody of the invention preferably contains one or no isoleucine or leucine amino acids within the last 18 amino acids of the C-terminus of the heavy chain of the antibody.

Preferably, the antibody contains, at the C-terminus end of the heavy chain, the sequence:

(Xaa₁)ₘ C(Xaa₂)ₙ

where: C=cysteine residue
   Xaa₁=independently any amino acid with the proviso that it is not I or L or forms a consecutive sequence X₁ X₂ X₃ V S X₄
      where: X₁═N, H or L
         X₂═V or Y
         X₃═S or N
         X₄=aliphatic amino acid
   Xaa₂=independently any amino acid, especially Y or A
   m=an integer of at least 2
   n=an integer of 0 to 5, preferably 1.

Antibodies capable of binding J-chain peptides are also provided, the antibodies comprising at their C-terminal end the sequence:

(Xaa₁)ₘ C(Xaa₂)ₙ

where: C=cysteine residue
   Xaa₁=independently any amino acid with the proviso that it is not I or L or forms a consecutive sequence X₁ X₂ X₃ V S X₄
      where: X₁═N, H or L
         X₂═V or Y
         X₃═S or N
         X₄=aliphatic amino acid
   Xaa₂=independently any amino acid, preferably Y or A
   m=at least 2
   n=0 to 5, especially 1.

Preferred synthetic tailpieces formed by such sequences include:

(a) SCMVGHEALPMNFTQKTIDRLSGKPACY
(b) SCMVGHEALPMNFTQKTIDRLSGKPAAACY
(c) SCMVGHEALPMNFTQKTIDRLSGKPHASTPEPDPVACY
and
(d) SCMVGHEALPMNFTQKTIDRLSGKPAAAAACY

Modifying the C-terminal end still further to include PAAAAACY at the C-terminus produced an improvement in antibody secretion.

Further improvement is expected by removing the C-terminal tyrosine residue and optionally replacing it with Alanine. The terminal tyrosine is expected to be a potential vacuolar targeting signal. Frigerio, L., et al. (The Plant Cell, Vol. 10 (1998), pages 1031-1042) looked at phaseolin, a legume protein in plants. Deletion of four C-terminal residues (Ala Phe Val Tyr) was found to prevent vacuolar targeting. Unpublished results by the authors of this paper also found that at least 50% of the vacuolar targeting was due to the C-terminal tyrosine residue.

It is also noted that other vacuolar sorting sequences comprising a C-terminus tyrosine residue have also been identified in other plant proteins such as albumin from rape and Arabidopsis (see Matsuoka K. and Neuhaus J. M., J. Exp. Bot., Vol. 50 (1999), pages 165-174).

Hence, more preferably the antibody comprises a C-terminus sequence selected from:

PAAAAACA
and
PAAAAAC.

Hence, preferably the antibody does not comprise a C-terminal tyrosine residue.

The antibodies of the invention may be used in the manufacture of medicaments to treat disease. Alternatively, they may be administered to a patient to treat disease or administered as prophylaxis.

The antibodies in the invention may also be used, for example, in the production of an assay or other techniques known to use antibodies.

Oligonucleotides for use in the manufacture of the antibodies of the invention are also provided. These include: 5′-ccatcgatggaatggacctgggttttt-3′, 5′-ccctctagactagtagc ataggccatc-3′, 5′-actgtagacaattccgccacctcagcctaca-3′, 5′-tgtaggctgaggtggcggaattgtctac agt-3′, 5′-gagcagctcaacagegattccgctcagtcag-3′, 5′-ctgactgagcggaaaacgctgttgagctgctc-3′, 5′-ttgcccatgaacttcgtccagaagaccatcga-3′, 5′-tcgatggtatctggacgaagttcatgggcaa-3′, 5′-aaaccc accaatgtcgctgtgtctgtgatcatg-3′, 5′-catgatcacagacacagcgacattggtgggttt-3′, 5′-aaacccaccaatg tcgctgtgtctgtgatcatg-3′, 5′-catgatcacagacacagcgacattggtgggttt-3′, 5′-ccctctagactatttacccgac agacggtc-3′, 5′-gagcagctcaacagcgttttccgctcagtcag-3′.

The invention will now be described by way of example only, with reference to the following figures:

FIG. 1. Schematic representation of the constructs used in this study.

All constructs were fused downstream of the CaMV35S promoter in the expression vector pJLH38. SP, signal peptide. The hearts indicate the glycosylation sites in the heavy chains.

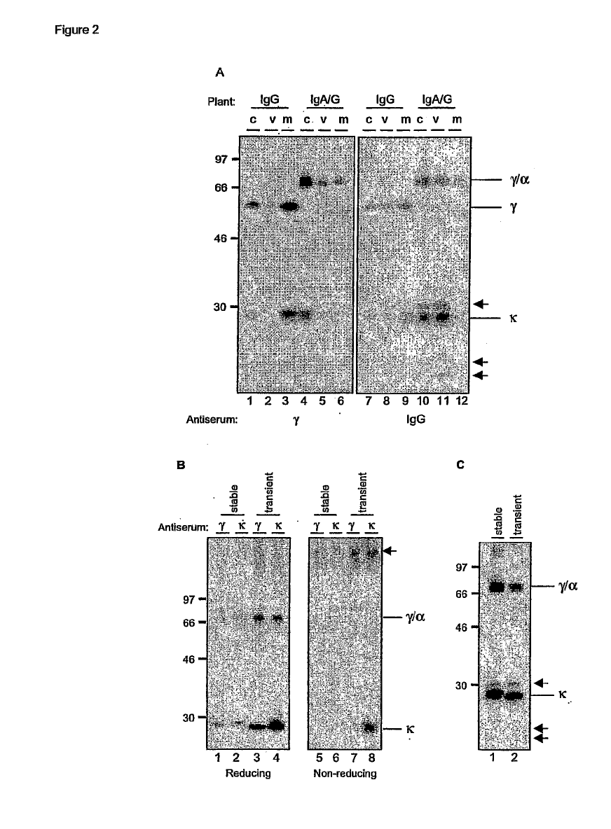

FIG. 2. Assembly and intracellular fate of transiently expressed Ig chains.

Tobacco mesophyll protoplasts were transfected with plasmids encoding k and α or k and γ/α chains, respectively. Cells were labelled for 16 hr with 35S methionine and cysteine. Total cell homogenates (c), purified vacuoles (v) or incubation media (m) were immunoprecipitated with anti gamma or anti IgG antisera. Polypeptides were visualised by SDS-PAGE and fluorography.

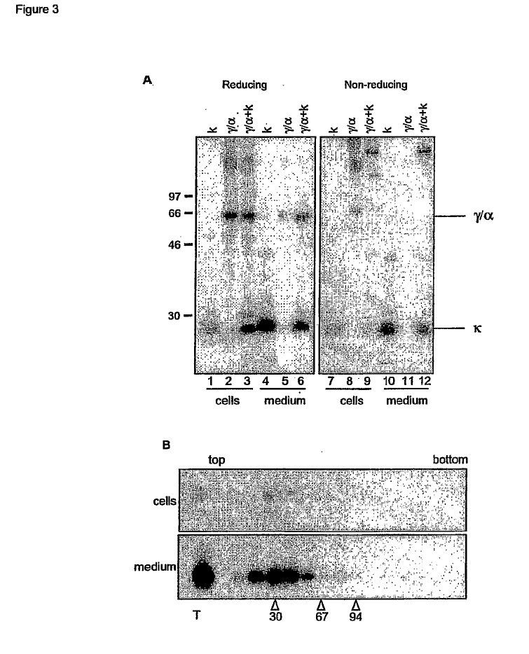

FIG. 3. Unassembled light chain is secreted as a monomer.

A. Protoplasts were transfected with plasmids encoding k chain, γ/α heavy chain or both chains together and labelled for 16 h. Cell homogenates and incubation media were immunoprecipitated with anti IgG and proteins resolved by reducing or non-reducing SDS-PAGE and fluorography.

B. Protoplasts were transfected with plasmid encoding k chain and labelled for 16 h. The cell homogenate and incubation medium were subjected to sedimentation velocity centrifugation on a linear 5-25% sucrose gradient. Gradient fractions were immunoprecipitated with anti IgG antiserum and polypeptides resolved by SDS-PAGE and fluorography. T, total (unfractionated) sample. Numbers at the bottom indicate molecular mass markers in kilodaltons.

FIG. 4. IgAJG travels through the Golgi complex.

A. Protoplasts were transfected with plasmids encoding k chain and γ/α heavy chain (wt), g/a lacking the two C-terminal glycosylation sites (Δ3,4) or γ/α lacking all four glycosylation sites (Δ1,2,3,4), respectively. Cells were pulse-labelled for 16 hours, homogenized and immunoprecipitated with anti IgG antiserum. Proteins were visualised by SDS-PAGE and fluorography.

B. Cells were transfected with plasmids encoding k and g/a chains and treated as in A. Immunoprecipitates were subjected to treatment with endoglycosydase H (endo H) or buffer (control) prior to SDS-PAGE analysis. The arrows indicate the position of vacuolar fragmentation products. The asterisk indicates the position of the k light chain. Numbers at left indicate molecular mass markers in kilodaltons.

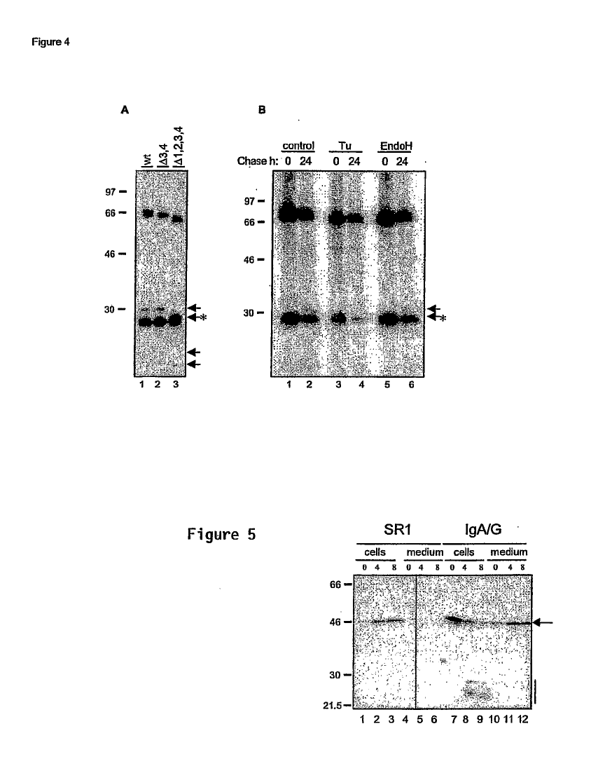

FIG. 5. Vacuolar delivery of IgA/G is not caused by stress.

Protoplasts from untransformed, wild type (SR1) or transgenic tobacco plants expressing IgA/G or IgG were transiently transfected with plasmids encoding phaseolin Δ418. Cells were labelled for 1 hr with 35S methionine and cysteine and chased for the indicated periods of time. Total cell homogenates were immunoprecipitated with anti phaseolin antiserum and polypeptides were visualised by SDS-PAGE and fluorography. The arrowhead indicates the position of intact phaseolin. The vertical bar in panel B indicates the position of vacuolar fragmentation products of phaseolin. Numbers at left indicate molecular mass markers in kilodaltons.

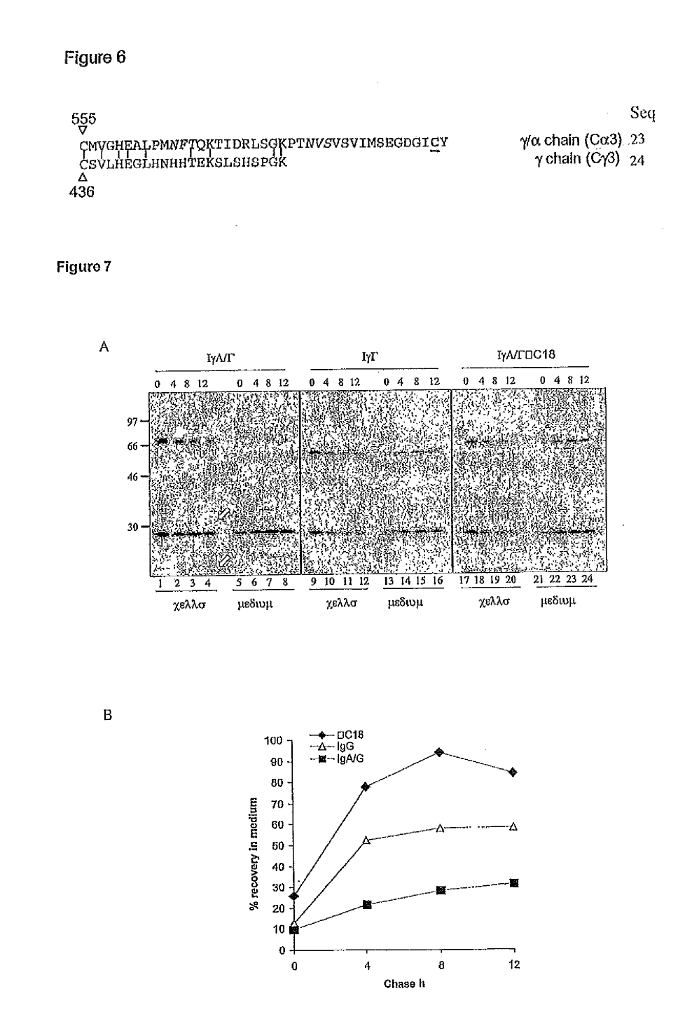

FIG. 6. Comparison of the C-terminal constant domains of IgA and IgG.

Comparison of the C-terminal tailpieces of the constant domains of IgA/G and IgG heavy chains. Amino acid sequence alignment was generated using the MegAlign program (DNASTAR Inc., Madison, USA). Vertical bars identify identical amino acid residues The putative N-glycosylation sites in γ/α are italicised The J-chain binding cysteine residue is underlined.

FIG. 7. Deletion of the Cα3 tailpiece results in secretion of IgA/G.

A. Tobacco protoplasts were transfected with plasmids encoding k chain and γ/α, γ or γ/αΔC18 heavy chains, respectively. Cells were pulse labelled for 1 h and chased for the indicated periods of time. Cell homogenates and incubation media were immunoprecipitated with anti IgG antiserum. Proteins were visualised by SDS-PAGE and fluorography. Numbers at left indicate molecular mass markers in kilodaltons. White arrows indicate vacuolar fragmentation products.

B. The fluorograms shown in A were subjected to densitometry to quantify the amount of secreted heavy chains, which accurately reflects the secretion of assembled antibody. Secreted heavy chains are expressed as percentage of total intracellular heavy chains immunoselected at 0 h chase.

FIG. 8. Sequence homology of the C-terminus ends of IgA and IgM heavy chains.

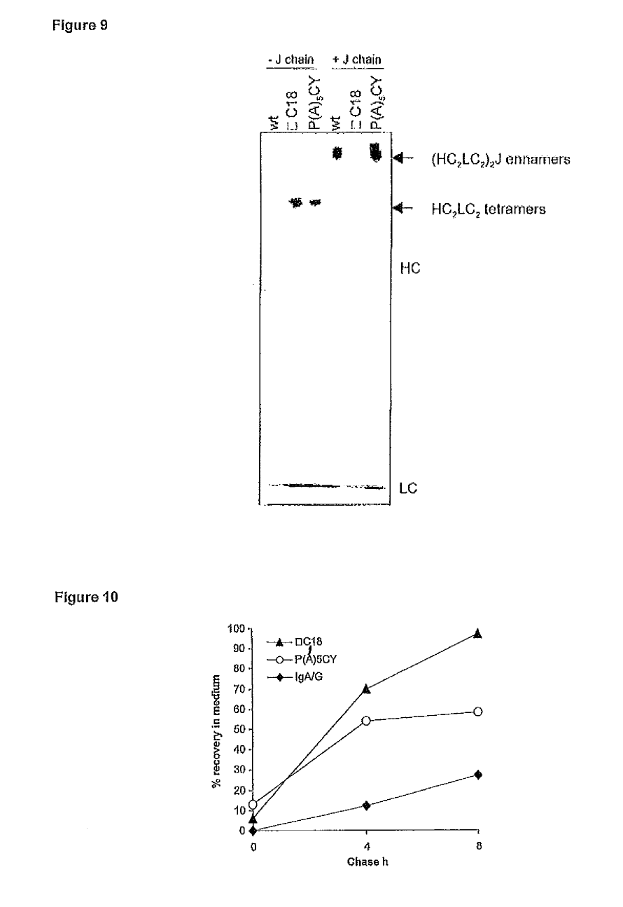

FIG. 9. An artificial C-terminal tail PAAAAACY allows J chain binding and dIgA/G ennamer assembly.

Tobacco protoplasts were transfected with plasmids encoding k chain and γ/α, γ/αΔC18 or γ/αΔC18P(A)₅CY heavy chains, respectively, either in the presence or in the absence of plasmid encoding the J chain. Cells were pulse labelled for 1 h. Cell homogenates were immunoprecipitated with anti IgG antiserum. To analyse their assembly state, proteins were resolved by non-reducing SDS-PAGE and visualised by fluorography. Note that the efficiency of ennamer assembly of P(A)₅CY is comparable to the wild-type IgA/G. ΔC18 is incapable of binding the J chain as it lacks the C-terminal cysteine.

FIG. 10. The artificial P(A)₅CY tail allows for improved antibody secretion.

Tobacco protoplasts were transfected with plasmids encoding k chain, J chain and γ/α, γ/αΔC18 or γ/αΔC18P(A)₅CY heavy chains. Cells were pulse labelled for 1 h and chased for the indicated periods of time. Cell homogenates and incubation media were immunoprecipitated with anti IgG antiserum. Proteins were visualised by reducing SDS-PAGE and fluorography. The fluorograms were subjected to densitometry to quantify the amount of secreted heavy chains. Secreted heavy chains are expressed as percentage of total intracellular heavy chains immunoselected at 0 h chase. Note that at 8 hours, recovery of P(A)₅CY in the medium is 2.3-fold higher than recovery of IgA/G.

### MATERIALS AND METHODS

Transgenic Plants

Transgenic Nicotiana tabacum cv Xanthi plant lines expressing assembled IgG and IgA/G under the control of the cauliflower mosaic virus 35S-promoter have previously been described. For protoplast transfection experiments, wild-type N. tabacum cv Petit Havana SR1 was used Plants were grown in axenic conditions under a 12 hour light-dark regime.

Recombinant DNA

All DNA manipulations were performed using established procedures.

The full length IgA/G g/a heavy chain was amplified from the binary vector pMON530 using the polymerase chain reaction. The oligonucleotides 5′-ccatcgatggaatggacctgggttttt-3′ and 5′-ccctctagactagtagcataggccatc-3′ containing ClaI and XbaI restriction sites before the start codon and after the stop codon for cloning purposes, were used. The digested PCR products were ligated into a pUC-based vector downstream of the CaMV35S-promoter (Denecke et al., 1992). The resulting plasmid was designated pJLH38.

The glycosylation site mutations were produced using the ‘Quickchange’ in vitro mutagenesis system (Stratagene, La Jolla, Calif.). Potential glycan sites mutations Ser 76 to Ala (Dglycan1 pJLH40), Thr 289 to Val (Dglycan2 pJLH41), Thr 526 to Ala (Dglycan3 pJLH42) and Ser 541 to Ala (Dglycan4 pJLH43) were introduced using the oligonucleotides 5′-actgtagacaattccgccacctcagcctaca-3′, 5′-tgtaggctgaggtggcggaattgtctacagt-3′, 5′-gagcagctcaacagcgttttccgctcagtcag-3′, 5′-ctgactgagcggaaaacgctgttgagctgctc-3′, 5′-ttgcccatgaacttcgtccagaagaccatcga-3′, 5′-tcgatggtcttctggacgaagttcatgggcaa-3′, 5′-aaacccaccaatgtcgctgtgtctgtgatcatg-3′ or 5′-catgatcacagacacagcgacattggtgggttt-3′ respectively, using pJLH38 as a template. Multiple glycan mutants D3,4 was also produced using Quickchange in vitro mutagenesis system (Stratagene, La Jolla, Calif.) with the oligonucleotides 5′-aaacccaccaatgtcgctgtgtctgtgatcatg-3′ and 5′-catgatcacagacacagcgacattggtgggttt-3′ and pJLH42 as template. The glycan mutant pJLH45, containing no glycosylation sites (D1,2,3,4) was produced by isolating the ClaI-NcoI fragment from pJLH40, the NcoI-EcoRI fragment pJLH41, EcoRI-XbaI fragment from pJLH44 and ligating them into the pUC vector previously cut with ClaI and XbaI.

Removal of the last 18 amino acids of the g/a heavy chain (DC18 pJLH47) was achieved by PCR using the anti-sense oligonucleotide 5′-ccctctagactatttacccgacagacggtc-3′ producing a stop codon followed by an XbaI site at position 537 with the sense oligo 5′-gagcagctcaacagcgttttccgctcagtcag-3′. The resulting PCR product was cut with EcoRI and XbaI and ligated into the expression vector cut with ClaI and XbaI along with a ClaI-EcoRI fragment of pJLH38.

Phaseolin expression constructs T343F and D418 are described in Pedrazzini et al. (1997) and Frigerio et al. (1998), respectively.

Protoplast Transfection

Protoplasts prepared from axenic leaves of tobacco (Nicotiana tabacum cv Petit Havana SRI) grown in sterile in vitro conditions under a 12 hour light-dark regime, were subjected to polyethylene glycol-mediated transfections as described by Predrazzini et al. (1997). 40 mg of each plasmid was used to transform 106 protoplasts in 1 ml. When only single antibody chains were expressed, the total amount of DNA was maintained constant among samples by adding 40 mg of empty expression vector pDHA. After transfection, cells were incubated at 25° C. before metabolic labelling.

In vivo labelling of protoplasts and analysis of expressed polypeptides.

Pulse-chase experiments were conducted by labelling protoplasts using ProMix (a mixture of 35S-Met and 35S-Cys; Amersham) and chasing with an excess of cold amino acids for the times stated (Predrazzini et al. 1997). After harvesting at desired time points, protoplasts and incubation media were frozen and homogenised by adding two volumes of ice-cold homogenisation buffer (150 mM Tric-HCl, 150 mM NaCl, 1.5 mM EDTA, and 1.5% (w/v) Triton X-100, pH 7.5) supplemented with Complete protease inhibitor cocktail (Roche). Immunoprecipitation of expressed polypeptides was performed as described previously (Frigerio et al. 1998) using rabbit polyclonal antisera raised against mouse IgG (whole molecule, Sigma), g heavy chain, k light chain (Southern Biotechnology), or bean phaseolin (Pedrazzini et al. 1997). Digestion of immunoprecipitated proteins proteins with endoglycosidase H (Roche) was performed as described previously (Ceriotti et al. 1991). For the analysis of the secreted free kappa light chains by sedimentation velocity, after radioactive labelling and homogenation cells, homogenates and incubation media, were loaded on top of a continuous 5 to 25% (w/v) linear sucrose gradient made in 150 mM NaCl, 1 mM EDTA, 0.1% Triton X-100, and 50 mM Tris-Cl, pH7.5. Samples were centrifuged at 39,000 rpm in a SW40 Ti rotor (Beckman Instruments, Inc., Fullerton, Calif.) for 20 hours at 20° C. K light chain was then immunoselected from each gradient fraction. Immunoselected proteins were resolved by 15% (w/v) non-reducing or reducing SDS-PAGE and revealed by fluorography.

Results

The inventors have previously shown that the intracellular fate of decaineric, secretory IgA/G is the same as its constituent tetrameric IgA/G sub-unit. They therefore decided to focus their efforts on the latter for simplicity of analysis. Tetrameric IgA/G consists of two K light chains and two hybrid IgA/G chains (FIG. 1). The hybrid heavy chain (denominated γ/α) consists of the IgG γ variable domain and constant Cγ1 and Cγ2 domains from monoclonal IgG Guy's 13, fused to the constant Cα2 and Cα3 domain from a secretory IgA. The Cα3 domain contains the C-terminal cysteine that is responsible for binding the J chain and contains regions necessary for contact with the secretory component. The additional Cα2 domain was initially added to provide an extra affinity tag, to facilitate purification of the antibody from plant tissue. The presence or the absence of this domain does not affect the trafficking of the molecule, and the inventors' unpublished observations.

In order to demonstrate that the fate of IgA/G molecules from transgenic plants differs from that of the parent IgG molecules, protoplasts obtained from transgenic plants expressing either IgG or IgA/G were subjected to pulse labelling for 16 hr with ³⁵S methionine and cysteine and subsequent vacuole purification. Total cell homogenates, purified vacuoles or incubation media were immunoprecipitated with anti ã antiserum. The unbound proteins were then re-immunoprecipitated with anti whole molecule IgG antiserum and polypeptides were revealed by SDS-PAGE and fluorography (FIG. 2A). The comparison of IgG and IgA/G shows that whilst IgG heavy and light chains are mainly recovered in the protoplast incubation medium after 16 hr (FIG. 2, lane 3), IgA/G is mostly found inside the cells and partially in purified vacuoles (lanes 4 and 5). Anti γ recognises only the constant gamma domains in the heavy chains. FIG. 2A shows that this antiserum coselects both heavy chains (γ for IgG and γ/α for IgA/G) and κ light chains, indicating that the light chains have assembled with the parental and recombinant heavy chains. Re-immunoprecipitation with anti IgG, that recognises the whole IgG molecule (and therefore most of the IgA/G molecule as well, excluding the alpha constant domains in γ/α) reveals the presence of additional, smaller IgA/G polypeptides in the total cell homogenates (FIG. 2A, lanes 10 and 11). These polypeptides are found predominantly in the vacuolar fraction and represent degradation products. This provides confirmation of their previous findings that IgA/G assembles efficiently but is secreted very slowly, with a conspicuous proportion of the molecules being delivered to the vacuole where they are ultimately degraded.

To understand the relative contribution of the heavy and light chains to the intracellular fate of IgA/G, the inventors decided to use transient expression in tobacco protoplasts. This technique has been successfully used to study the behaviour and trafficking of proteins in the plant secretory pathway, although it has never been used for complex multimeric proteins. The transient expression system allows rapid testing of mutant constructs and would therefore be a valuable tool for the analysis of potential of trafficking mutants of IgA/G. In order to reliably use this technique in this study the inventors wanted to prove that the behaviour of the Ig chains was comparable in transiently and permanently transformed tobacco cells. To achieve these aims they cloned the Ig coding sequences (heavy and light chains, FIG. 1), fused to the CaMV35S promoter, into a CaMV35S-driven vector for transient expression. Tobacco mesophyll protoplasts were transfected with plasmids encoding k chain and IgA/G γ/α heavy chain. Transfected cells or cells obtained from transgenic IgA/G plants were metabolically labelled for 2 hr with 35S methionine and cysteine and homogenised. Homogenates were immunoprecipitated with either anti γ or anti κ antisera. As previously mentioned, anti γ recognises only the constant γ domains in the heavy chains, whereas anti κ binds to the light chain only. Both of these antisera were capable of co-selecting both heavy and light chains, indicating assembly of the IgA molecule after transient and permanent expression (FIG. 2B, lanes 1-4). Assembly is very efficient, as further confirmed by the fact that the molecules migrate with the mobility expected for fully assembled heterotetramers when resolved on non-reducing SDS-PAGE (FIG. 2B, lanes 5-8) with no free heavy chain detected in either transient of stably expressing protoplasts. An excess of free light chain can be observed in lane 8 and will be discussed further below. The discrepancy observed in the mobility of the k chain between transient and stable expression is due to the fact that a different signal peptide was used to prepare the transient expression construct compared to the murine signal peptide used to generate transgenic plants and see Methods) As the original sequence contains additional residues between the predicted signal peptide cleavage site and the first codon of mature κ chain, this probably results in a slightly different electrophoretic mobility.

The inventors' observations in transgenic plants reveal that the IgA/G tetramer is partially delivered to vacuoles. This results in the appearance of degradation products after a long chase (FIG. 2A). To test whether this is also the case for transiently expressed IgA/G, they compared the phenotype of the molecules after a 16 hr metabolic labelling (FIG. 2C). Subsequent immunoprecipitation with anti IgG, which recognises the whole IgG molecule, reveals the presence of small polypeptides resulting from degradation (FIG. 2C) when the IgA/G molecule is expressed transiently. The sizes of these fragments are comparable in both transiently and stably expressed antibody chains. They therefore conclude that fragments observed in transient expression are also the result of vacuolar delivery. Therefore, both the assembly and intracellular transport events of IgA/G are faithfully reproduced in transient expression using tobacco mesophyl protoplasts.

From FIG. 2B (lane 8) and their previous observations in transgenic plants it appears that in cells expressing heavy and light chains (or other chains in addition to them), an excess of free, unassembled light chain can be detected. This also seems to be the case with transiently expressed chains. This excess is reflected by an increased amount of light chain in the medium, suggesting that the free light chain be secreted. To test this, the inventors transiently transformed tobacco protoplasts with plasmid encoding the k chain, the γ/α heavy chain, or both chains together and subjected them to metabolic labelling for 16 hours. Protoplasts were then homogenised and immunoprecipitated with anti IgG. FIG. 3A shows that immunoprecipitation with anti IgG antiserum reveals the presence of a larger amount of κ light chains compared to heavy chain in both the cells and the incubation medium. When the samples are resolved on non-reducing SDS-PAGE (FIG. 3A, lanes 7-12) it appears evident that the excess κ light chain observed is free κ light chain not associated with heavy chain. In mammalian cells there is some evidence that free unassembled light chain can be secreted as covalent or non-covalent dimer. From the experiment in non-reducing conditions (FIG. 3A, lanes 10 and 12) it is clear that there are no disulfide-linked dimers of κ. To determine if non-covalent dimerisation occurs prior to secretion of k chain in plants, they transfected protoplasts with k chain and metabolically labelled them overnight. They then loaded cell homogenates and incubation medium onto linear 5-25% sucrose gradients and subjected them to sedimentation velocity centrifugation, along with molecular mass markers. Gradient fractions were then immunoprecipitated with anti IgG antiserum (FIG. 3B). Clearly, the vast majority of secreted light chain is retrieved in fractions of mass around 30 kDa, a size compatible with the monomeric form. It was concluded that ê is secreted as a monomer in tobacco mesophyll protoplasts.

In previous studies the inventors have shown that transport of IgA/G to the vacuole can be inhibited by the fungal metabolite Brefeldin A. They used that evidence to conclude that IgA/G was transported through the Golgi complex. Recently, however, it has become clear that there exist routes from the plant ER to the vacuole, which, although prone to BFA inhibition, seem to bypass the Golgi. In the light of this new evidence it is clear that BFA inhibition alone can no longer be used as a sole evidence for transport through the Golgi complex. Thus, in order to test whether IgA/G does indeed travel through the Golgi they have studied the state of one of its N-linked glycans.

One prominent IgA/G vacuolar degradation product migrates above the κ chain around 30 kDa. This fragment is comprised of the variable and constant gamma domains and in non-reducing conditions is associated with the κ light chain to yield the Fab fragment (data not shown). When all glycosylation sites in γ/α are abolished (D1,2,3,4, FIG. 4A), the 30 kDa fragment seems to disappear (FIG. 4A, lane 3). The same phenotype is observed when wild-type heavy chain is expressed in the presence of tunicamycin, an inhibitor of N-linked glycosylation (FIG. 4B, compare lanes 1-2 with lanes 3-4). The observed disappearance of such fragment upon tunicamycin treatment or mutation of the glycosylation sites is likely due to the fact that the mobility of the becomes very similar to that of the κ chain, resulting in co-migration of the two polypeptides. The fact that the mobility shift is only detectable in the mutant γ/α chain where all four putative glycosylation sites have been removed (FIG. 4A, lane 3) but not in the mutant chain carrying mutations in the two sites located in the α domain (D3,4, FIG. 4A, lane 2) provides further evidence that the 30 kDa fragment originates from the γ domain of the hybrid γ/α heavy chain.

As fragmentation occurs in the vacuolar compartment it was expected that the glycan present on the degradation product carry a complex (i.e. Golgi-modified) glycan if the molecule has travelled through the Golgi complex en route to the vacuole. If this is the case, this glycan should be resistant to endoglycosidase H (endo H) digestion. Endo H resistance is a reliable indication that a glycoprotein has travelled through the medial and trans-Golgi complex. Indeed, treatment with endo H after transient expression, metabolic labelling and subsequent immunoprecipitation, does not affect the mobility of the 30 kDa fragment (FIG. 4B, compare lanes 2 and 6) indicating that this glycan has acquired endo H resistance. It is however clear that the enzyme is active, as the mobility of the heavy chain is increased by endo H treatment at the end of the pulse, similarly to what observed in the presence of tunicamycin (FIG. 4B, compare lanes 1, 3 and 5). This is expected as, shortly after synthesis, whilst still in the ER, the heavy chain glycans are in the high-mannose, endo H-sensitive form.

Based on the results shown, it was therefore concluded that transport of IgA/G to the vacuole occurs through the Golgi complex.

Is the observed vacuolar delivery of a proportion of SIgA/G the result of quality control or, rather, the result of stress imposed to the secretory system by the over-expression of this heterologous protein? Could vacuolar targeting be the effect of saturation of secretion? This has never been reported, although the opposite can occur: over-expression of vacuolar proteins can lead to their partial secretion [Frigerio, 1998]. To test this possibility, cells from plants expressing the parent IgG or SIgA/G were transfected with plasmid encoding a secreted form of the storage protein phaseolin, Δ418. This phaseolin mutant is normally secreted very efficiently after visiting the Golgi complex. If expression of the antibody is saturating the ER capacity and imposing stress onto the system to the point of reducing secretion, it would be expected that secretion of Δ418 would be inhibited as well. However, FIG. 5 shows that in the antibody-expressing cells, the fate of phaseolin is not affected and the protein is still secreted with the same efficiency observed in wild-type cells. It is therefore concluded that vacuolar delivery of IgA cannot be attributed to stress to the endomembrane system.

To further explore the possibility of a spurious effect due to stress on the secretory system, the inventors wanted to test whether expression of IgA/G was affecting transport of a normally vacuolar protein. They used the well studied phaseolin variant T343F, which is transported to the vacuole in tobacco protoplasts. Phaseolin undergoes proteolytic fragmentation in the vacuole, so the appearance of fragments is indicative of vacuolar delivery. In the antibody-expressing cells, phaseolin still delivered to vacuoles, with efficiency comparable to wild-type cells. A proportion of phaseolin molecules is secreted in the incubation medium. They have previously shown that this is due to saturation of the sorting machinery during transient expression In IgA/G expressing cells, there is no shift in the ration vacuolar/secreted phaseolin. This indicates that expression—and vacuolar delivery—of IgA/G is not competing with vacuolar sorting of phaseolin, suggesting that the two proteins may be sorted to the vacuole by different mechanisms.

The work presented so far shows that the IgA/G tetrameric molecule is transported to the vacuolar compartment via the Golgi complex where it is ultimately degraded. As this intracellular fate differs dramatically to the fate of the parental IgG molecule and the inventors have ruled out the possibility of trafficking events occurring as a result of ER stress, the most likely explanation for IgA/G delivery to the vacuole is the existence of positive, albeit cryptic, sorting information within the molecule. Clearly, this signal must be absent from the parent IgG as this is secreted efficiently. γ and γ/α heavy chains differ in that γ/α has two additional, constant a domains. The inventors believe that the signal must be exposed and they therefore compared the C-terminal regions of the two heavy chains. Sequence alignments of the IgG γ3 domain and the IgA/G α3 domains reveals that the α3 domain is 18 residues longer than the γ3 (FIG. 6). This extension constitutes the tailpiece containing the cysteine residue required for J chain binding. This tailpiece is rich in hydrophobic residues, a feature typical of C-terminal vacuolar sorting signals (ctVSS). Given the heterogeneous nature of ctVSS, the inventors believe that the tailpiece is recognised as a vacuolar targeting signal within plant cells. To test this hypothesis, they deleted the 18-residue C-terminal tail to produce a truncated heavy γ/α chain, denominated ΔC18. They co-transfected protoplasts with plasmids encoding the κ light chain and γ/α, γ or ΔC18 heavy chains, respectively, and subjected them to pulse-chase analysis. When co-expressed with light chain, ΔC18 assembled correctly (not shown) and was secreted with kinetics comparable to IgG (FIG. 6A). Note that whilst the low extracellular amount of IgA/G chains remains constant, and is probably the result of cell breakage during sample handling before homogenisation, the amount of secreted IgA/G containing DC18 heavy chains increases steadily with time, with kinetics comparable to the secretion of the IgG chains (FIG. 7, compare lanes 13-16 with lanes 21-24). The increased secretion of DC18 was paralleled by a decrease in the appearance of vacuolar degradation fragments, as revealed by overexposing immunoprecipitates after a long labelling (FIG. 6B). This shows that deletion of the C-terminal 18 amino acids of the IgA/G heavy chain results in reduced vacuolar targeting and increased secretion of the assembled tetramers. Therefore the inventors concluded that the C-terminal tail of the hybrid γ/α chain is responsible for vacuolar delivery of IgA/G.

Discussion

Plants are unique among non-animal systems in their ability to assemble complex multimeric proteins. In this work the inventors have studied the intracellular fate of a hybrid secretory immunoglobulin IgA/G and gained further insight on how plants respond to the expression of a complex heterologous protein. The efficiency of IgA/G assembly in the plant ER is virtually 100%. In this respect, the plant ER is a very versatile and efficient folding compartment for foreign proteins, as shown by other studies. Similarly to mammalian cells, the molecular chaperone BiP is involved in binding unassembled heavy chains and it is released upon co-expression of the companion light chains (Nuttall and Frigerio, unpublished results).

Single Chains

Single light chains have a different fate. Plants secrete free or unassembled κ chain with kinetics typical of ‘normal’ secretory proteins. In contrast with animal cells, where single light chains can either be retained in the ER, degraded, or secreted as disulfide-bonded dimers, unassembled κ is quantitatively secreted as a monomer.

Transport through the Golgi

With regards with synthesis, translocation into the ER and quality control, the inventors' findings indicate that plants are perfectly happy to produce large amounts of antibody. The ER is not stressed, indeed it is able to accept much more heterologous protein. As for anterograde transport, their previous observations in transgenic plants showed that transport of sIgA/G to the vacuole can be inhibited by brefeldin A treatment. Recently, however, the inventors have demonstrated that BFA can inhibit vacuolar transport of a protein that does not travel through the Golgi. To provide final evidence for the involvement of the Golgi in the transport of IgA/G the inventors have therefore analysed the state of one of its N-linked glycans. This glycan clearly acquires endoglycosidase H resistance after a chase, confirming glycan processing in the Golgi.

Vacuolar Delivery

For plant-made IgA/G, the problems start post-assembly and, presumably, post-Golgi. A large proportion of the molecules, which are expected to be secreted, are actually transported to the vacuole where they slowly undergo proteolytic fragmentation. The inventors have shown that this is not a stress response from the plant but the result of cryptic signalling.

C-Ter Tail Sequence and Sorting to Vacuoles

It remains to be seen whether this signal is recognised by a low affinity receptor or if targeting occurs via different mechanism. The inventors have previously shown that IgA/G does not interact with microsomal membranes. At present it is known that IgA/G and phaseolin do not compete for sorting (FIG. 5B). Whether this is due to different targeting mechanisms or to a high capacity of the sorting machinery remains to be established.

Biotechnological Perspectives

The ultimate goal of secretory antibody expression in plants is to produce large amounts of protein in a way that allows for easy purification. Protein secretion by the roots is a most promising approach. Maximising SIgA/G secretion is therefore an attractive goal. The inventors have identified the signal that prevents a vast proportion of the immunoglobulin molecules from being secreted. Deletion of this signal leads to hybrid IgA/G secretion at levels comparable to IgG secretion, indicating that no further intracellular processes exist to prevent exocytosis. This is the first essential step towards the production of a fully secreted complex immunoglobulin. The inventors are aware that complete deletion of the C-terminal tail, which also contains the C-terminal cysteine involved in J chain binding, is too drastic as it also prevents assembly of the decameric molecule.

Homology studies of IgM heavy chains show high levels of homology with IgA C-terminal ends. Hence, antibodies containing IgM heavy chains are also expected to have similar secretion problems.

The inventors have noted that deletion of the C-terminal end of the heavy chains of IgA and IgM results in deletion of the C-terminal cysteine residue responsible for J-chain beinding. The presence of the J-chain increases peptidase resistance and stability of the antibody. This can be restored by adding an artificial peptide chain to the end of the antibody to restore J-chain binding ability. The peptide chain lacks the secondary structure encoding the sites that block secretion in native antibody heavy chains.

Synthetic tailpieces (underlined) which are expected to work in transfected tobacco cells include:

(a) SCMVGHEALPMNFTQKTIDRLSGKPACY
(b) SCMVGHEALPMNFTQKTIDRLSGKPAAACY
(c) SCMVGHEALPMNFTQKTIDRLSGKPHASTPEPDPVACY

Ig heavy chains containing synthetic tailpieces will be assayed for their ability to bind J chain and to afford secretion of assmebled, multimeric antibodies. This will be initially established by transient gene expression in tobacco mesophyll protoplasts. Plasmids encoding modified heavy chains, light chains and J chain were co-transfected into protoplasts. Assembly of the (Heavy₂Light₂)₂J complex and secretion of the complex were analysed in vivo by pulse-chase analysis, followed by immunoprecipitation with specific antisera, reducing or non-reducing SDS-PAGE and fluorography.

FIGS. 9 and 10 show that using the -PAAAAACY tailpiece as the C-terminus end of the heavy chain, whilst the efficiency of ennamer assembly of -PAAAAACY is comparable to the wild-type IgA/G, recovery of -PAAAAACY in the medium was 2.3-fold higher than wild-type IgA/G.

Further improvements are expected by deletion of the C-terminal tyrosine and optionally replacing it by alanine.

### REFERENCES

(1) Daniell, H, et al. (2001), Trends Plant Sci., Vol. 6, pages 219-226.

(2) Ma, J K-C, et al. (1995), Science, Vol. 268, pages 716-719.

(3) Frigerio, L, et al. (2000), Plant Physiol., Vol. 123, pages 1483-1493.

(4) Matsuoka, K, Nakamura, K. (1999), Plant Mol. Biol., Vol 41, pages 825-835.

(5) Vitale, A, Raikhel, N V. (1999), Trends Plant Sci., Vol. 4, pages 149-155.

(6) Ma, J K-C, et al. (1994), Eur. J. Immunol., Vol. 24, pages 131-138.

(7) Tabe, L, Higgins, T J V. (1998), Trends Plant Sci., Vol. 3, pages 282-286.

(8) Mestecky, J, McGhee, J. (1987), Adv. Immunol., Vol. 40, pages 153-245.

(9) Törmäkangas, K, et al. (2001), Plant Cell, Vol. 13, pages 2021-2032.

(10) Phillipson, B A, et al. (2001), Plant Cell, Vol 13, pages 2005-2020.

(11) Frigerio, L, et al. (1998), Plant Cell, Vol. 10, pages 1031-1042.

(12) Frigerio, L, et al. (1998), J. Biol. Chem., Vol. 273, pages 14194-14199.

(13) Denecke, J, et al. (1992), EMBO J., Vol. 11, pages 2345-2355.

(14) Leitzgen, K, et al. (1997), J. Biol. Chem., Vol. 272, pages 3117-3123.

(15) Frigerio, L, et al. (2001), Plant Cell, Vol. 13, pages 1109-1126.

(16) Vitale, A, Chrispeels, M J (1984), J. Cell Biol., Vol. 99, pages 133-140.

(17) Mattu, T S, et al. (1998), J. Biol. Chem., Vol. 273, pages 2260-2272.

(18) Crofts, A J, et al. (1999), Plant Cell, Vol. 11, pages 2233-2247.

(19) Denecke, J, et al. (1990), Plant Cell, Vol. 2, pages 51-59.

(20) Borisjuk, N V, et al. (1999), Nat. Biotechnol., Vol. 17, pages 466-469.

1. A method of making an antibody molecule, the antibody containing an immunoglobulin heavy chain comprising a α3 domain or a mu domain, the method comprising:
(a) Providing a nucleotide sequence encoding the immunoglobulin heavy chain;
(b) Modifying the nucleotide sequence in the region of the nucleotide sequence encoding the C-terminus 18 amino acids of the completed heavy chain to remove, or reduce the effectiveness of, one or more vacuolar targeting signal sequences to form a modified nucleotide sequence;
(c) Inserting the modified nucleotide sequence into a host cell; and
(d) Causing the host cell to express the modified nucleotide sequence to form the modified antibody heavy chain and secrete the modified antibody heavy chain from the host cell.

2. A method according to claim 1 wherein the heavy chain molecule is IgA, IgM or an IgA/G hybrid.

3. A method according to claim 1, wherein nucleotide sequence is modified by one or more point mutations of the nucleotide sequence, deleting one or more nucleotides, adding one or more nucleotides and/or replacing one or more nucleotides with a synthetic nucleotide sequence.

4. A method according to claim 3, wherein the synthetic nucleotide sequence encodes an amino acid sequence of general formula:
(Xaa₁)ₘ C(Xaa₂)ₙ
where: C=a cysteine residue
Xaa₁=independently any amino acid with the proviso that it is not from I, L or forms a consecutive sequence X₁ X₂ X₃ V S X₄
where: X₁═N, H or L
X₂═V or Y
X₃═S or N
X₄=aliphatic amino acid
Xaa₂=independently any amino acid
m=at least 2
n=0 to 5.

5. A method according to claim 4, wherein Xaa₂ is Y and n=1.

6. A method according to claim 1, wherein nucleotides encoding the last 16 amino acids of the heavy chain are deleted.

7. A method according to claim 1, wherein one nucleotides encoding an amino acid sequence having a formula:
(a) SCMVGHEALPMNFTQKTIDRLSGKPACY,
(b) SCMVGHEALPMNFTQKTIDRLSGKPAAACY,
(c) SCMVGHEALPMNFTQKTIDRLSGKPHASTPEPDPVACY
or
(d) SCMVGHEALPMNFTQKTIDRLSGKPAAAAACY

8. A method according to claim 1, wherein the nucleotide sequence modified originally encoded the amino acid sequence:
X₁ X₂ X₃ V S X₄
where: X₁═N, H or L
X₂═V or Y
X₃═S or N
X₄=aliphatic amino acid.

9. A method according to claim 8, wherein the amino acid sequence is: N V S V S V.

10. A method according to claim 1, wherein the nucleotide sequence modified encoded L or I.

11. A method according to claim 10, wherein the modified amino acid is one or both of an isoleucine 3 amino acids and/or 10 amino acids from the C-terminus end of the completed heavy chain.

12. A method according to claim 1, wherein the nucleotide sequence modified is within the sequence:
P T X₁ X₂ X₃ V S X₄ X₅ X₆ X₇ X₈ X₉ X₁₀ X₁₁ X₁₂ C X₁₃
where: X₁═N, H or L, preferably N
X₂═V or Y, preferably V
X₃═S or N
X₄=an aliphatic amino acid, preferably V or L
X₅=an aliphatic amino acid, preferably I, V or L
X₆=M, V or L, especially M
X₇═S or A
X₈=E or D
X₉=any amino acid, preferably G, V, A or T
X₁₀=D, E, G or A, preferably D
X₁₁=G or S, preferably G
X₁₂═I, T, V, Z or A, preferably I or T
X₁₃=may or may not be present and, where present is A or Y

13. A method of adding J-chain binding capability to the heavy chain of an antibody comprising the steps of:
(a) providing a nucleotide encoding an immunoglobulin heavy chain;
(b) adding to that nucleotide a nucleotide sequence encoding a synthetic tail with the amino acid sequence:
(Xaa₁)ₘ C(Xaa₂)ₙ
where: C=Cys
Xaa₁ is independently any amino acid with the proviso that it is not I or L or forms a consecutive sequence X₁ X₂ X₃ V S X₄ (where X₁═N, H or L; X₂═V or Y; X₃═S or N; X₄=aliphatic amino acid)
Xaa₂=independently any amino acid
m=at least 2
n=0 to 5; and
(c) expressing the completed nucleotide in a host cell to form an immunoglobulin heavy chain capable of binding J-chain.

14. A method according to claim 1, wherein the host cell is a plant cell.

15. A method according to claim 14, wherein the plant cell is part of a transgenic plant.

16. A method according to claim 1, additionally comprising the step of isolating and purifying the antibody molecule.

17. A method according to claim 16, wherein the antibody is subjected to a protease digest to for Fab or F(ab′)₂ fragments,

18. An antibody obtainable by a method according to claim 1.

19. An antibody containing a heavy chain comprising an α3 domain or a mu domain, the α3 domain or mu domain lacking one or more targeting signals towards the C-terminal end.

20. An antibody capable of binding J-chain comprising at its C-terminal end the sequence:
(Xaa₁)ₘ C(Xaa₂)ₙ
where: C=Cys
Xaa₁ is independently any amino acid with the proviso that it is not I or L or forms a consecutive sequence X₁ X₂ X₃ V S X₄ (where X₁═N, H or L; X₂═V or Y; X₃═S or N; X₄=aliphatic amino acid)
Xaa₂=independently any amino acid
m=at least 2
n=0 to 5

21. An antibody according to claim 18 which does not contain the targeting signal: X₁ X₂ X₃ V S X₄
where: X₁═N, H or L
X₂═V or Y
X₃═S or N
X₄=aliphatic amino acid.

22. An antibody according to claim 21, wherein the targeting signal is N V S V S V.

23. An antibody according to claim 18 which contains one or no isoleucine or leucine amino acids within the last 18 amino acids at the C-terminus of the heavy chain of the antibody.

24. An antibody according to claim 18 comprising at the C-terminus end of the heavy chain of antibody, the sequence:
(Xaa₁)ₘ C(Xaa₂)ₙ
where: C=a cysteine residue
Xaa₁=independently any amino acid with the proviso that it is not I or L or forms a consecutive sequence X₁ X₂ X₃ V S X₄
where: X₁═N, H or L
X₂═V or Y
X₃═S or N
X₄=aliphatic amino acid
Xaa₂=independently any amino acid
m=at least 2
n=0 to 5.

25. An antibody according to claim 1 in which at least two, preferably two to four, glycine or alanine residues are present downstream of a C-terminal targeting sequence

26. An antibody according to claim 1 in which at least the terminal amino acid residue of a C-terminal targeting sequence is replaced by at least two, preferably two to four, glycine or alanine residues.

27. Use of an isolated antibody according claim 18 in the manufacture of a medicament to treat disease.

28. A method of treating a disease by administering an antibody according to claim 18 to a patient.

29. A method of prophylaxis, comprising administering an antibody according to claim 18 to a person or animal.

30. A vector comprising a nucleotide sequence encoding an antibody according to claim 18.

31. A host cell comprising a nucleotide sequence encoding antibody according to claim 18 or a vector according to claim 30.

32. A transgenic plant comprising a nucleotide encoding an antibody according to claim 18.

33. An immunoassay comprising an antibody as defined in claim 18.


SEQUENCE LISTING (72 sequences)

SEQ ID NO: 1 - protein, 6 aa, Artificial sequence
  Modified targeting signal in the final antibody heavy chain
  misc_feature (1)..(1): Asn, His or Leu
  misc_feature (2)..(2): Val or Tyr
  misc_feature (3)..(3): Ser or Asn
  misc_feature (6)..(6): An aliphatic amino acid, especially Val or Leu
XXXVSX

SEQ ID NO: 2 - protein, 6 aa, Artificial sequence
  Modified targeting signal in the final antibody heavy chain
NVSVSV

SEQ ID NO: 3 - skipped

SEQ ID NO: 4 - protein, 6 aa, Artificial sequence
  Modified targeting signal in the final antibody heavy chain
  misc_feature (1)..(1): Asn, His or Leu
  misc_feature (2)..(2): Val or Tyr
  misc_feature (3)..(3): Ser or Asn
  misc_feature (6)..(6): Any aliphatic amino acid
XXXVSX

SEQ ID NO: 5 - protein, 18 aa, Artificial sequence
  Modified targeting signal in the final antibody heavy chain
  misc_feature (3)..(3): Asn, His or Leu. Preferably Asn
  misc_feature (4)..(4): Val or Tyr, preferably Val
  misc_feature (5)..(5): Ser or Asn
  misc_feature (8)..(8): An aliphatic amino acid, preferably Val or Leu
  misc_feature (9)..(9): An aliphatic amino acid, preferably Ile, Val or Leu
  misc_feature (10)..(10): Met, Val or Leu, preferably Met
  misc_feature (11)..(11): Ser or Ala
  misc_feature (12)..(12): Asp or Glu
  misc_feature (13)..(13): Any amino acid, preferably Gly, Val, Ala or Thr
  misc_feature (14)..(14): Asp, Glu, Gly, or Ala, preferably Asp
  misc_feature (15)..(15): Gly or Ser, preferably Gly
  misc_feature (16)..(16): Ile, Thr, Val, Glx or Ala, preferably Ile or Thr
  misc_feature (18)..(18): May be present or absent. When present, it is Ala or Tyr. Most preferably Ala.
PTXXXVSXXXXXXXXXCX

SEQ ID NO: 6 - protein, 6 aa, Artificial sequence
  Modified targeting signal in the final antibody heavy chain
  misc_feature (1)..(1): Asn, His or Leu
  misc_feature (2)..(2): Val or Tyr
  misc_feature (3)..(3): Ser or Asn
  misc_feature (6)..(6): Aliphatic amino acid
XXXVSX

SEQ ID NO: 7 - protein, 28 aa, Artificial sequence
  Modified targeting signal in the final antibody heavy chain
SCMVGHEALPMNFTQKTIDRLSGKPACY

SEQ ID NO: 8 - protein, 30 aa, Artificial sequence
  Modified targeting signal in the final antibody heavy chain
SCMVGHEALPMNFTQKTIDRLSGKPAAACY

SEQ ID NO: 9 - protein, 38 aa, Artificial sequence
  Modified targeting signal in the final antibody heavy chain
SCMVGHEALPMNFTQKTIDRLSGKPHASTPEPDPVACY

SEQ ID NO: 10 - DNA, 27 bp, Artificial sequence
  Synthetic oligonucleotide
ccatcgatggaatggacctgggttttt

SEQ ID NO: 11 - DNA, 27 bp, Artificial sequence
  Synthetic oligonucleotide
ccctctagactagtagcataggccatc

SEQ ID NO: 12 - DNA, 31 bp, Artificial sequence
  Synthetic oligonucleotide
actgtagacaattccgccacctcagcctaca

SEQ ID NO: 13 - DNA, 31 bp, Artificial sequence
  Synthetic oligonucleotide
tgtaggctgaggtggcggaattgtctacagt

SEQ ID NO: 14 - DNA, 32 bp, Artificial sequence
  Synthetic oligonucleotide
gagcagctcaacagcgttttccgctcagtcag

SEQ ID NO: 15 - DNA, 32 bp, Artificial sequence
  Synthetic oligonucleotide
ctgactgagcggaaaacgctgttgagctgctc

SEQ ID NO: 16 - DNA, 32 bp, Artificial sequence
  Synthetic oligonucleotide
ttgcccatgaacttcgtccagaagaccatcga

SEQ ID NO: 17 - DNA, 32 bp, Artificial sequence
  Synthetic oligonucleotide
tcgatggtcttctggacgaagttcatgggcaa

SEQ ID NO: 18 - DNA, 33 bp, Artificial sequence
  Synthetic oligonucleotide
aaacccaccaatgtcgctgtgtctgtgatcatg

SEQ ID NO: 19 - DNA, 33 bp, Artificial sequence
  Synthetic oligonucleotide
catgatcacagacacagcgacattggtgggttt

SEQ ID NO: 20 - DNA, 30 bp, Artificial sequence
  Synthetic oligonucleotide
ccctctagactatttacccgacagacggtc

SEQ ID NO: 21 - DNA, 32 bp, Artificial sequence; identical to SEQ ID NO: 14
  Synthetic oligonucleotide

SEQ ID NO: 22 - protein, 8 aa, Artificial sequence
  Tailpiece of artificial C-terminus of heavy chain
PAAAAACY

SEQ ID NO: 23 - protein, 40 aa, Mus musculus
CMVGHEALPMNFTQTIDRLSGKPTNVSVSVIMSEGDGICY

SEQ ID NO: 24 - protein, 23 aa, Mus musculus
CSVLHEGLHNHHTEKSLSHSPGK

SEQ ID NO: 25 - protein, 25 aa, Mus musculus
IDRLSGKPTNVSVSVIMSEGDGICY

SEQ ID NO: 26 - protein, 25 aa, Mus musculus
IDRLSGKPTNVSVSVIMSEGDGICY

SEQ ID NO: 27 - protein, 25 aa, Mus musculus
IDRLSGKPTNVSVSVIMSEGDGICY

SEQ ID NO: 28 - protein, 25 aa, Mus musculus
IDRLSGKPTNVSVSVIMSEGDGICY

SEQ ID NO: 29 - protein, 25 aa, Mus musculus
IDRLSGKPTNVSVSVIMSEGDGICY

SEQ ID NO: 30 - protein, 25 aa, Mus musculus
IDRLSGKPTNVSVSVIMSEGDGICY

SEQ ID NO: 31 - protein, 25 aa, Mus musculus
IDRLSGKPTNVSVSVIMSEGDGICY

SEQ ID NO: 32 - protein, 25 aa, Mus musculus
IDRLSGKPTNVSVSVIMSEGDGICY

SEQ ID NO: 33 - protein, 25 aa, Mus musculus
IDRLSGKPTNVSVSVIMSEGDGICY

SEQ ID NO: 34 - protein, 25 aa, Mus musculus
IDRLSGKPTNVSVSVIMSEGDGICY

SEQ ID NO: 35 - protein, 25 aa, Mus musculus
IDRLSGKPTNVSVSVIMSEGDGICY

SEQ ID NO: 36 - protein, 25 aa, Mus musculus
IDRLSGKPTNVSVSVIMSEGDGICY

SEQ ID NO: 37 - protein, 25 aa, Mus musculus
IDRLSGKPTNVSVSVIMSEGDGICY

SEQ ID NO: 38 - protein, 25 aa, Mus musculus
IDRLSGKPTNVSVSVIMSEGDGICY

SEQ ID NO: 39 - protein, 25 aa, Mus musculus
IDRLSGKPTNVSVSVIMSEGDGICY

SEQ ID NO: 40 - protein, 25 aa, Mus musculus
IDRLSGKPTNVSVSVIMSEGDGICY

SEQ ID NO: 41 - protein, 25 aa, Mus musculus
IDRLSGKPTNVSVSVIMSEGDGICY

SEQ ID NO: 42 - protein, 25 aa, Mus musculus
IDRLSGKPTNVSVSVIMSEGDGICY

SEQ ID NO: 43 - protein, 25 aa, Homo sapiens
IDRLAGKPTHVNVSVVMAEVDGTCY

SEQ ID NO: 44 - protein, 25 aa, Gorilla gorilla
IDRLAGKPTHVNVSVVMAEVDGTCY

SEQ ID NO: 45 - protein, 25 aa, Homo sapiens
IDRLAGKPTHVNVSVVMAEVDGTCY

SEQ ID NO: 46 - protein, 25 aa, Homo sapiens
IDRLAGKPTHVNVSVVMAEVDGTCY

SEQ ID NO: 47 - protein, 25 aa, Homo sapiens
IDRLAGKPTHVNVSVVMAEVDGTCY

SEQ ID NO: 48 - protein, 25 aa, Homo sapiens
IDRLAGKPTHVNVSVVMAEVDGTCY

SEQ ID NO: 49 - protein, 25 aa, Homo sapiens
IDRLAGKPTHVNVSVVMAEVDGTCY

SEQ ID NO: 50 - protein, 25 aa, Homo sapiens
IDRLAGKPTHVNVSVVMAEVDGTCY

SEQ ID NO: 51 - protein, 25 aa, Homo sapiens
IDRLAGKPTHVNVSVVMAEVDGTCY

SEQ ID NO: 52 - protein, 25 aa, Homo sapiens
IDRLAGKPTHVNVSVVMAEVDGTCY

SEQ ID NO: 53 - protein, 25 aa, Homo sapiens
IDRLAGKPTHVNVSVVMAEVDGTCY

SEQ ID NO: 54 - protein, 25 aa, Homo sapiens
IDRLAGKPTHVNVSVVMAEVDGTCY

SEQ ID NO: 55 - protein, 25 aa, Oryctolagus cuniculus
IDRLAGKPTHVNVSVVVADVEAVCY

SEQ ID NO: 56 - protein, 25 aa, Mesocricetus auratus
VDRSTGKPTLYNVSLIMSDAGGTCY

SEQ ID NO: 57 - protein, 25 aa, Mus musculus
VDKSTGKPTLYNVSLIMSDTGGTCY

SEQ ID NO: 58 - protein, 25 aa, Homo sapiens
VDKSTGKPTLYNVSLVMSDTAGTCY

SEQ ID NO: 59 - protein, 25 aa, Homo sapiens
VDKSTGKPTLYNVSLVMSDTAGTCY

SEQ ID NO: 60 - protein, 25 aa, Homo sapiens
VDKSTGKPTLYNVSLVMSDTAGTCY

SEQ ID NO: 61 - protein, 25 aa, Homo sapiens
VDKSTGKPTLYNVSLVMSDTAGTCY

SEQ ID NO: 62 - protein, 25 aa, Homo sapiens
VDKSTGKPTLYNVSLVMSDTAGTCY

SEQ ID NO: 63 - protein, 25 aa, Homo sapiens
VDKSTGKPTLYNVSLVMSDTAGTCY

SEQ ID NO: 64 - protein, 25 aa, Canis familiaris
VDKSTGKPTLYNVSLVLSDTAGTZY

SEQ ID NO: 65 - protein, 25 aa, Suncus murinus
VDKTSGKPTLYNVSLVLSDTASTCY

SEQ ID NO: 66 - protein, 25 aa, Oryctolagus cuniculus
VDKSTGKPTLYNVSLIMSDTASTCY

SEQ ID NO: 67 - protein, 25 aa, Gallus gallus
VDRASGKASAVNVSLVLADSAAACY

SEQ ID NO: 68 - protein, 22 aa, Ralstonia solanacearum
RIGGPPTGITSDVYLSVYEGVC

SEQ ID NO: 69 - protein, 32 aa, Artificial sequence
  Modified targeting signal in the final antibody heavy chain
SCMVGHEALPMNFTQKTIDRLSGKPAAAAACY

SEQ ID NO: 70 - protein, 4 aa, Artificial Sequence
  c-terminal targeting residues of antibody
AFVY

SEQ ID NO: 71 - protein, 8 aa, Artificial sequence
  Modified c-terminal targeting signal of the antibody
PAAAAACA

SEQ ID NO: 72 - protein, 7 aa, artificial sequence
  Modified c-terminal targeting signal of the antibody
PAAAAAC